(19) United States
(12) Patent Application Publication
(10) Pub. No.: US 2026/0114754 A1
(43) Pub. Date: Apr. 30, 2026

(54) Acoustic Technique for Characterizing Propensity for Missing Fundamental Illusion

(71) Applicant: Board of Regents, The University of Texas System, Austin, TX (US)

(72) Inventors: Eric A. Freudenthal, El Paso, TX (US); Kevin J. Moreno, El Paso, TX (US); Natasha Rovelli, El Paso, TX (US)

(21) Appl. No.: 19/376,187

(22) Filed: Oct. 31, 2025

Related U.S. Application Data
(60) Provisional application No. 63/714,663, filed on Oct. 31, 2024.

(51) Int. Cl. A61B 5/12; A61B 5/00

(52) U.S. Cl. CPC A61B 5/123; A61B 5/4064; A61B 5/7405

(57) ABSTRACT

Diagnosing the perception of missing fundamental illusion is provided. The method comprises generating a first tone comprising a sinusoidal sum of harmonic multiples of a first fundamental frequency. The first tone does not include the first fundamental frequency. A second tone is generated comprising a sinusoidal sum of a second fundamental frequency with a number of harmonics multiples of the second fundamental frequency. The second fundamental frequency is sufficiently higher than the first fundamental frequency to be distinguished as a different pitch by a user. The harmonic multiples of the first fundamental frequency and second fundamental frequency are the same multiples. The first tone and second tone are presented to a user in a predetermined sequence unknown to the user. An answer input is received from the user regarding which of the first tone or second tone is higher. The user is diagnosed according to the answer input.

### CROSS-REFERENCE TO RELATED APPLICATION

This application claims the benefit of U.S. Provisional Patent Application Ser. No. 63/714,663, filed Oct. 31, 2024, and entitled “Acoustic Technique for Characterizing Propensity for Missing Fundamental Illusion,” herein incorporated by reference in its entirety.

### BACKGROUND INFORMATION

### 1. Field

The present disclosure relates to auditory perception, and more specifically to identifying individuals with reduced susceptibility to the missing fundamental illusion.

### 2. Background

The missing fundamental illusion is an auditory phenomenon wherein a person perceives a pitch that is not actually present in sound waves. Certain overtones (a.k.a harmonics) of a fundamental frequency are heard, but the actual fundamental frequency, F0, is absent or significantly attenuated.

Despite this missing F0, the brain fills in the missing information and creates the perception of a dominant F0 pitch whose frequency is the greatest common factor (GCF) of the other frequencies in an acoustic tone. Most humans and some other species of animals are prone to this illusion.

### SUMMARY

An illustrative embodiment provides a method of diagnosing the perception of missing fundamental illusion. The method comprises generating a first tone comprising a sinusoidal sum of a number of harmonic multiples of a first fundamental frequency. The first tone does not include the first fundamental frequency. A second tone is generated comprising a sinusoidal sum of a second fundamental frequency with a number of harmonics multiples of the second fundamental frequency. The second fundamental frequency is sufficiently higher than the first fundamental frequency to be distinguished as a different pitch by a listener with normal hearing, e.g., 5% is typically sufficient. The harmonic multiples of the first fundamental frequency and second fundamental frequency are the same multiples. The first tone and second tone are presented to a user in a sequence unknown to the user. An answer input is received from the user regarding which of the first tone or second tone is higher. The user is diagnosed according to the answer input.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 depicts a diagram of a tone generator and selector in accordance with an illustrative embodiment;

FIG. 2 depicts a diagram of a timing generator in accordance with an illustrative embodiment;

FIG. 3 depicts a flowchart illustrating a process for diagnosing the ability to identify fundamental frequencies in speech in accordance with an illustrative embodiment; and

FIG. 4 depicts a block diagram of a data processing system in accordance with an illustrative embodiment.

### DETAILED DESCRIPTION

The illustrative embodiments recognize and take into account that missing fundamental illusion creates the perception of a loud F0 pitch whose frequency is the greatest common factor (GCF) of the other frequencies in an acoustic tone.

The illustrative embodiments recognize and take into account that voiced phonemes comprise harmonic series containing a number of components at different frequencies.

The GFC frequency of a harmonic series is commonly referred to as the fundamental frequency (F0), H1, natural/intonation frequency, or timbre (in music theory). F0 in voice utterances refers to the lowest frequency of a periodic waveform produced during speech. It sets the base tone or pitch of the voice. It corresponds to the rate at which the vocal cords vibrate when air passes through them. In speech analysis, F0 determines tonal aspects of speech, intonation patterns, and stress. In English, modulation of F0 primarily conveys mood and emotions, and may also serve as a feature exploited by the brain in the parsing of language.

Harmonic series also comprise harmonics, also known as overtones, which are integer multiples of the fundamental frequency, F0. Harmonics will occur at frequencies of 2F0, 3F0, 4F0, etc. Higher harmonics add richness and complexity to the sound but do not change the pitch. The relationship between F0 and higher harmonics is foundational to the way complex sounds, including voice, are produced and perceived.

The loudness of F0 in voice may be significantly lower than other harmonics present in the voice, or even deleted by sound reproduction systems. Intent and mood are substantially communicated through modulation of F0. Therefore, the ability to perceive F0 in voice, even when attenuated, is necessary to discern mood and intent in verbal communication.

Neurodiverse individuals with attention and autistic-related conditions (henceforth called Autism Spectrum Disorder or ASD) tend to more accurately perceive sounds, meaning they are less susceptible to the missing fundamental illusion of hearing the GCF frequency (F0) in a vocal harmonic.

In contrast, individuals with allistic (neurotypical) acoustic perception are more susceptible to missing fundamental illusion. This illusion (which distorts their perception of sounds) adaptively provides heightened social awareness due to the ability to “fill in” F0 and thereby discern mood and intention in verbal communication.

The illustrative embodiments provide a method for characterizing an individual's susceptibility to missing fundamental illusion (MFI). The illustrative embodiments play a sequence of sounds comprising at least two harmonic series with differing F0 frequencies. The harmonic series with the higher F0 actually contains its F0 frequency. In contrast, the harmonic series with the lower F0 does not contain its F0 frequency (i.e. F0 is missing from that harmonic series). The test subject then tries to identify which tone has the higher or lower pitch. If the test subject perceived the tone with the lower F0 as being lower (despite the missing F0), the test subject perceives the MFI.

When examining subjects'pitch perception in speech, it may be desirable to play a sequence whose cadence and shift approximate normal speech patterns conventionally used to communicate mood or intent.

It is expected that the combinations of harmonics required to perceive the illusion accurately will vary among people and generally require more harmonics for people with ASD-related conditions. The time to respond to these questions may be longer for people with ASD-related conditions.

The method of the illustrative embodiments is non-invasive and can be implemented using commodity devices such as, e.g., tablet computers, phones, laptop computers, etc.). This testing method can be implemented within a short time frame (e.g., 5 minutes) and requires minimal training. The method of the illustrative embodiments is applicable to people with language skills that can describe perceived acoustic pitches, e.g., most 6+ year olds with normal hearing and language skills.

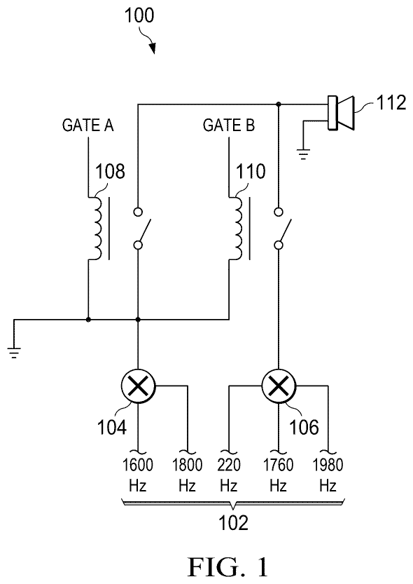

FIG. 1 depicts a diagram of a tone generator and selector in accordance with an illustrative embodiment. Tone generator 100 can be implemented in hardware, software, or a combination of both.

Tone generator 100 comprises a number of frequency sources 102. In the present example, the frequency signals generated by frequency sources 102 include 1600 HZ, 1800 Hz, 220 Hz, 1760 Hz, and 1980 Hz.

Summing junctions 104 and 106 sum different frequency signals to produce respective harmonic series A and B. Harmonic series A has a fundamental (aka natural) frequency (F0) of 200 Hz but does not contain this fundamental frequency. It is created by summing sinusoidal signals at 1600 Hz and 1800 Hz, which are the eighth and nineth harmonics of the (missing) fundamental frequency.

Harmonic series B is a harmonic series with a fundamental frequency (F0) of 220 Hz. It is created by summing sinusoidal signals at 220 Hz, 1760 Hz, and 1980 Hz, which are the fundamental frequency plus its eighth and nineth harmonics.

The signals for harmonic series A and B are gated through normally open relays to the speaker 112. The coils that convey signal currents for harmonic series A and harmonic series B are respectively labelled Gate A 108 and Gate B 110.

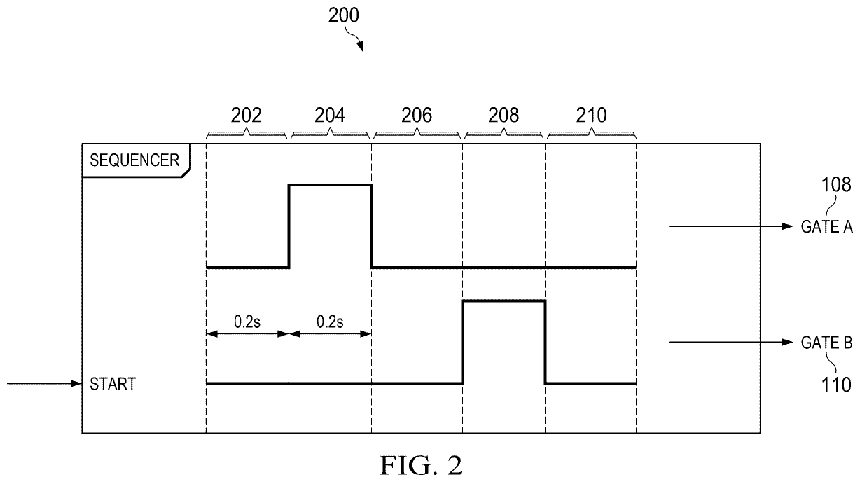

FIG. 2 depicts a diagram of a timing generator in accordance with an illustrative embodiment. Timing generator 200 controls sound emission from tone generator 100 by energizing relays that connect the speaker 112 to signals for harmonic series A or harmonic series B.

When the timing generator is inactive, Gate A 108 and Gate B 110 are both inactive, and no sound is emitted. In the present example, after a “start” signal triggers the timing generator 200 Gate A 108 and Gate B 110 are inactive for the first 0.2 second 202. Neither relay is energized, and no sound is emitted.

For the next 0.2 s 204, Gate A 108 is active, and Gate B 110 is inactive. Relay A is energized, and harmonic series A is emitted by the speaker 112.

For the following 0.2 s 206, Gate A 108 and Gate B 110 are both inactive. Neither relay is energized, and no sound is emitted.

For the next 0.2 s 208, Gate A is inactive, and Gate B 110 is active. Relay B is energized, and harmonic series B is emitted by the speaker 112.

Afterwards 210, Gate A 108 and Gate B 110 are both inactive. Neither relay is energized, and no sound is emitted.

The functionality of tone generator 100 and timing generator 200 can be implemented using a number of alternative methods. For example, alternative gating devices can be used instead of relays, such as a variable gain device that implements cosine fading.

Alternate timings and ordering of the single-tone phases may also be used. For example, the harmonic series with the lower (missing) fundamental frequency (F0) may be played before the harmonic with higher (present) fundamental frequency. In addition, durations other than 0.2 second may also be used for the harmonic series and the intervals between them. Additional tones from the harmonic series, with or without the presence of their natural frequency (F0), may also be used.

Tone sequences may imitate spoken prosody commonly used to convey an emotion or intention where inability to discern natural frequency would likely result in confusion for a different emotion or intention. For example, in English, questions commonly end with rising pitch.

Alternative harmonic series with different natural frequencies may also be used, as well as alternative harmonic series including different numbers of harmonics.

Speaker 112 might be replaced by or supplemented with alternative acoustic transducers such as, e.g., earphones, earbuds, ultrasonic interference devices, and bone vibration devices.

The method of the illustrative embodiments utilizes pairs of harmonic series referred to as “High” and “Low” tones. F0 of the High tone's harmonic series may be 5% higher than F0 of the Low tone. People with normal hearing can easily discriminate between high/low pitch for frequencies that differ by 5%. For example, one such pair would have F0 of the Low tone at 200 Hz, and F0 of the High tone at 210 Hz.

The contents of the tones are sums of sinusoids of equal amplitude at various frequencies. The fundamental frequency is included in the High tone but not in the Low tone. In one embodiment, both tones include the eighth and nineth harmonic, e.g., 1600 and 1800 Hz for the Low tone with F0 at 200 Hz and 1680 and 1990 Hz for the High tone with F0 at 210 Hz. Therefore, the Low tone is the sum of sinusoids at 1600 and 1800 Hz, and the High tone is the sum of sinusoids at 210, 1680, and 1890 Hz.

In this example implementation, all amplitudes of the constituent sinusoids are the same, and the Low and High tones are normalized to the same loudness. In other embodiments intended to confuse subjects without MFI, the amplitudes of the constituent sinusoids may not be equal.

People with a strong susceptibility to an accurate MFI will hear a loud tone at 200 Hz when exposed to the Low tone and a loud tone at 210 Hz when exposed to the High tone and will therefore readily identify “Low” as having a lower pitch than “High” despite the absence of F0 from the Low tone.

In contrast, people with no (or low) susceptibility to MFI will either hear the High tone as comprising only relatively high pitches (around 2 kHz) and the Low tone as comprising a low pitch (at 210 Hz) mixed with some higher pitches. Therefore, the person with no susceptibility to MFI will likely find it difficult to characterize one pitch as higher than the other or will identify the Low tone as having a higher pitch than the High tone.

A person who experiences an inaccurate MFI at a frequency higher than the Low tone (or even the High tone) may similarly find it difficult to characterize one pitch as higher or even identify the Low tone as having higher pitch than the High tone.

When used clinically, High/Low pairs of tones are presented in sequence. Subjects identify whether the second tone is higher or lower than the first tone and whether this determination was obvious. In order to achieve statistical robustness many pairs are presented to a user in varying (e.g., random) order with different pairs of fundamental frequencies, possibly all with the same frequency difference (e.g., 5%).

Because susceptibility to the MFI may vary among people and increase when more harmonics are present, the number of harmonics included in the High and Low tones is an adjustable test parameter. A test with varying numbers of harmonics can be used to determine the number of harmonics necessary for a subject to robustly perceive the MFI.

Susceptibility to MFI may also vary between long tones (typical of music) and staccato sequences of short tones (typical of language). Some people are better able to perceive the hidden fundamental frequency from faster tones, while others are better able to perceive it from longer tones. This difference may reflect differences in neurological function and perceptual deficits. If this is the case, evaluation using multiple timings of tone duration and time between tones can be used in differential diagnosis.

Alternate stimuli may be created with fundamental frequency patterns typical of spoken language. For example, a subject may be asked to discriminate between a tone sequence with a rising last tone (typical of a question in spoken English) from a tone sequence with a falling last tone (typical of an imperative statement), where each tone is a harmonic series as described above.

Empirical evidence has also demonstrated that susceptibility to MFI also depends on harmonic richness (the number of harmonics in a tone). People with impaired MFI are better able to generate an accurate phantom fundamental frequency (F0) as harmonic richness increases (i.e. more harmonics of F0 are added to a tone). Therefore, an individual with impaired MFI can perceive the missing F0 in a tone if enough harmonics are added to the harmonic series comprising that tone. The harmonic richness needed to achieve MFI in those with impaired ability will vary from person to person.

Furthermore, those with impaired MFI will initially perceive a missing F0 that is higher in pitch than the actual missing F0. For example, if the missing F0 for a tone is 200 Hz, the test subject may initially perceive a missing F0 of 210 Hz as harmonics are added to the tone. As additional harmonics are added to the tone, the perceived F0 will decrease until matching the true missing F0 of 200 Hz.

This phenomenon can be incorporated into the testing procedure of the illustrative embodiments. Using the example above, the test may begin with harmonic series A (comprising 1600 Hz and 1800 Hz, the eighth and nineth harmonics of the missing F0 of 200 Hz) and harmonic series B (comprising an F0 of 220 Hz and its eight and nineth harmonics of 1760 Hz and 1980 Hz). Initially, the test subject may not perceive a pitch difference between the tones comprising harmonic series A and B or may even perceive harmonic series A as having a higher pitch than harmonic series B. Over the course of several iterations, harmonic series A and B can be progressively enriched with additional harmonics (e.g., fifth, sixth, seventh harmonics, etc.) until the test subject begins to perceive a pitch difference between harmonic series A and B.

As explained above, as the harmonic series are progressively enriched, the test subject may initially perceive a phantom F0 for harmonic series A that is higher pitched than the actual missing F0 (e.g. 210 Hz instead of 200 Hz). As that harmonic series are further enriched, the test subject will eventually perceive the true missing F0 of harmonic series A. In this manner, the number of harmonics comprising a harmonic series (tone) can be used as another variable testing parameter.

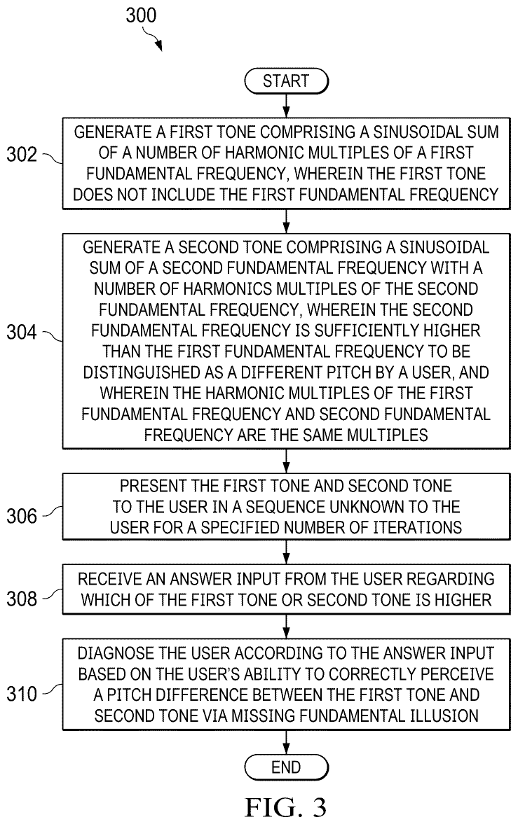

FIG. 3 depicts a flowchart illustrating a process for diagnosing the perception of missing fundamental illusion in accordance with an illustrative embodiment. Process 300 may be implemented with tone generator 100 and timing generator 200.

Process 300 begins by generating a first tone comprising a sinusoidal sum of a number of harmonic multiples of a first fundamental frequency (step 302). The first tone does not include the first fundamental frequency.

Process 300 then generates a second tone comprising a sinusoidal sum of a second fundamental frequency with a number of harmonics multiples of the second fundamental frequency (step 304). The second fundamental frequency is sufficiently higher than the first fundamental frequency to be distinguished as a different pitch by a user with normal hearing, e.g., 5% is typically sufficient. The harmonic multiples of the first fundamental frequency and second fundamental frequency are the same multiples such as the eighth and nineth harmonics. The harmonic multiples of the first fundamental frequency and second fundamental frequency may be the eighth and nineth harmonics. The number of harmonic multiples in the first tone and second tone is an adjustable test parameter used to determine the number of harmonics necessary for the user to perceive the first fundamental frequency missing from the first tone. The number of harmonic multiples in the first tone and second tone may be progressively increased over subsequent iterations.

The first tone and second tone may be normalized to the same loudness. Alternatively, the first tone and second tone may have different levels of loudness.

The first tone and second tone are presented to the user in a sequence unknown to the user for a specified number of iterations (step 306), and an answer input is received from the user regarding which of the first tone or second tone is higher (step 308). The first tone, second tone, and the interval between them have respective specified durations. The durations of the first and second tones may be equal or different and either or both can change over multiple iterations as can the duration of the interval between the first and second tones. Multiples timings of tone duration and time between the first and second tones can be used for differential diagnosis.

The frequency interval between the first tone and second tone can be varied to characterize the frequency of a perceived dominant frequency that is higher than the missing first fundamental frequency.

Whichever of the first or second tones is presented to the user last in the sequence may be presented as a rising tone or a falling tone.

The user is diagnosed according to the answer input based on the user's ability to correctly perceive a pitch difference between the first tone and second tone via missing fundamental illusion (step 310). Process 300 then ends. The steps of process 300 may be repeated a number of times with different first and second tones to ensure greater statistical rigor for diagnosing the user.

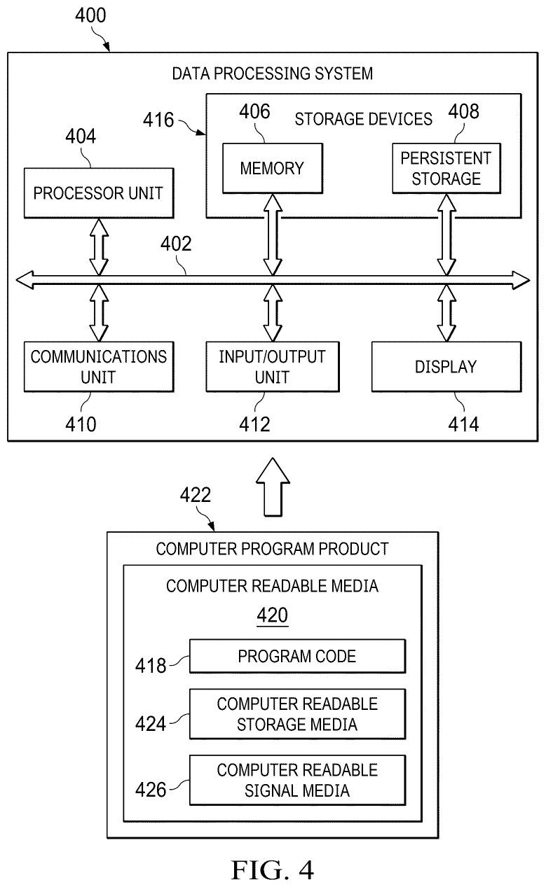

Turning now to FIG. 4, an illustration of a block diagram of a data processing system is depicted in accordance with an illustrative embodiment. Data processing system 400 may be used to implement tone generator 100 in FIG. 1 and/or timing generator 200 in FIG. 2 or portions thereof. In this illustrative example, data processing system 400 includes communications framework 402, which provides communications between processor unit 404, memory 406, persistent storage 408, communications unit 410, input/output (I/O) unit 412, and display 414. In this example, communications framework 402 takes the form of a bus system.

Processor unit 404 serves to execute instructions for software that may be loaded into memory 406. Processor unit 404 may be a number of processors, a multi-processor core, or some other type of processor, depending on the particular implementation. In an embodiment, processor unit 404 comprises one or more conventional general-purpose central processing units (CPUs). In an alternate embodiment, processor unit 404 comprises one or more graphical processing units (GPUs).

Memory 406 and persistent storage 408 are examples of storage devices 416. A storage device is any piece of hardware that is capable of storing information, such as, for example, without limitation, at least one of data, program code in functional form, or other suitable information either on a temporary basis, a permanent basis, or both on a temporary basis and a permanent basis. Storage devices 416 may also be referred to as computer-readable storage devices in these illustrative examples. Memory 406, in these examples, may be, for example, a random access memory or any other suitable volatile or non-volatile storage device. Persistent storage 408 may take various forms, depending on the particular implementation.

For example, persistent storage 408 may contain one or more components or devices. For example, persistent storage 408 may be a hard drive, a flash memory, a rewritable optical disk, a rewritable magnetic tape, or some combination of the above. The media used by persistent storage 408 also may be removable. For example, a removable hard drive may be used for persistent storage 408. Communications unit 410, in these illustrative examples, provides for communications with other data processing systems or devices. In these illustrative examples, communications unit 410 is a network interface card.

Input/output unit 412 allows for input and output of data with other devices that may be connected to data processing system 400. For example, input/output unit 412 may provide a connection for user input through at least one of a keyboard, a mouse, or some other suitable input device. Further, input/output unit 412 may send output to a printer. Display 414 provides a mechanism to display information to a user.

Instructions for at least one of the operating system, applications, or programs may be located in storage devices 416, which are in communication with processor unit 404 through communications framework 402. The processes of the different embodiments may be performed by processor unit 404 using computer-implemented instructions, which may be located in a memory, such as memory 406.

These instructions are referred to as program code, computer-usable program code, or computer-readable program code that may be read and executed by a processor in processor unit 404. The program code in the different embodiments may be embodied on different physical or computer-readable storage media, such as memory 406 or persistent storage 408.

Program code 418 is located in a functional form on computer-readable media 420 that is selectively removable and may be loaded onto or transferred to data processing system 400 for execution by processor unit 404. Program code 418 and computer-readable media 420 form computer program product 422 in these illustrative examples. In one example, computer-readable media 420 may be computer-readable storage media 424 or computer-readable signal media 426.

In these illustrative examples, computer-readable storage media 424 is a physical or tangible storage device used to store program code 418 rather than a medium that propagates or transmits program code 418. Computer readable storage media 424, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Alternatively, program code 418 may be transferred to data processing system 400 using computer-readable signal media 426. Computer-readable signal media 426 may be, for example, a propagated data signal containing program code 418. For example, computer-readable signal media 426 may be at least one of an electromagnetic signal, an optical signal, or any other suitable type of signal. These signals may be transmitted over at least one of communications links, such as wireless communications links, optical fiber cable, coaxial cable, a wire, or any other suitable type of communications link.

The different components illustrated for data processing system 400 are not meant to provide architectural limitations to the manner in which different embodiments may be implemented. The different illustrative embodiments may be implemented in a data processing system including components in addition to or in place of those illustrated for data processing system 400. Other components shown in FIG. 4 can be varied from the illustrative examples shown. The different embodiments may be implemented using any hardware device or system capable of running program code 418.

As used herein, the phrase “a number” means one or more. The phrase “at least one of”, when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

For example, without limitation, “at least one of item A, item B, or item C” may include item A, item A and item B, or item C. This example also may include item A, item B, and item C or item B and item C. Of course, any combinations of these items may be present. In some illustrative examples, “at least one of” may be, for example, without limitation, two of item A; one of item B; and ten of item C; four of item B and seven of item C; or other suitable combinations.

The flowcharts and block diagrams in the different depicted embodiments illustrate the architecture, functionality, and operation of some possible implementations of apparatuses and methods in an illustrative embodiment. In this regard, each block in the flowcharts or block diagrams may represent at least one of a module, a segment, a function, or a portion of an operation or step. For example, one or more of the blocks may be implemented as program code.

In some alternative implementations of an illustrative embodiment, the function or functions noted in the blocks may occur out of the order noted in the figures. For example, in some cases, two blocks shown in succession may be performed substantially concurrently, or the blocks may sometimes be performed in the reverse order, depending upon the functionality involved. Also, other blocks may be added in addition to the illustrated blocks in a flowchart or block diagram.

The description of the different illustrative embodiments has been presented for purposes of illustration and description, and is not intended to be exhaustive or limited to the embodiments in the form disclosed. Many modifications and variations will be apparent to those of ordinary skill in the art. Further, different illustrative embodiments may provide different features as compared to other illustrative embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

What is claimed is:

1. A method of diagnosing the perception of missing fundamental illusion, the method comprising:
generating a first tone comprising a sinusoidal sum of a number of harmonic multiples of a first fundamental frequency, wherein the first tone does not include the first fundamental frequency;
generating a second tone comprising a sinusoidal sum of a second fundamental frequency with a number of harmonics multiples of the second fundamental frequency, wherein the second fundamental frequency is sufficiently higher than the first fundamental frequency to be distinguished as a different pitch by a user, and wherein the harmonic multiples of the first fundamental frequency and second fundamental frequency are the same multiples;
presenting the first tone and second tone to the user in a predetermined sequence unknown to the user for a specified number of iterations;
receiving an answer input from the user regarding which of the first tone or second tone is higher; and
diagnosing the user according to the answer input based on the user's ability to correctly perceive a pitch difference between the first tone and second tone via missing fundamental illusion.

2. The method of claim 1, wherein the second fundamental frequency is at least five percent higher than the first fundamental frequency.

3. The method of claim 1, wherein first tone and second tone are normalized to a same loudness.

4. The method of claim 1, wherein the first tone and second tone have different levels of loudness.

5. The method of claim 1, wherein the number of harmonic multiples in the first tone and second tone is an adjustable test parameter used to determine the number of harmonics necessary for the user to perceive the first fundamental frequency missing from the first tone.

6. The method of claim 5, further comprising progressively increasing the number of harmonic multiples in the first tone and second tone over subsequent iterations.

7. The method of claim 1, wherein:
the first tone has a first duration;
the second tone has a second duration; and
an interval between the first and second tones has a third duration.

8. The method of claim 7, wherein at least one of the first duration, second duration, or third duration changes over subsequent iterations.

9. The method of claim 1, wherein whichever of the first tone or second tone is presented to the user last in the sequence is presented as a rising tone.

10. The method of claim 1, wherein whichever of the first tone or second tone is presented to the user last in the sequence is presented as a falling last tone.

11. The method of claim 1, further comprising varying a frequency interval between the first tone and second tone to characterize the frequency of a perceived dominant frequency that is higher than the missing first fundamental frequency.
